(19) United States
(12) Patent Application Publication
(10) Pub. No.: US 2019/0060601 A1
(43) Pub. Date: Feb. 28, 2019

(54) LIGHTING LAMPS GENERATING NOVEL AESTHETIC EXPERIENCES IN HUMANS AND OTHER BEINGS USING PHYSICAL INTERACTIVE SYSTEMS

(71) Applicant: Universidad de los Andes, Bogota D.C. (CO)

(72) Inventor: Rafael Hernando Barragan Romero, Bogota D.C. (CO)

(21) Appl. No.: 15/764,736

(22) PCT Filed: Sep. 30, 2016
(86) PCT No.: PCT/CO2016/000007

§ 371 Date: Oct. 8, 2018

(30) Foreign Application Priority Data
Sep. 30, 2015 (CO) 15231644

(51) Int. Cl. A61M 21/00

(52) U.S. Cl. CPC A61M 21/00; A61M 2021/0044; A61M 2205/3331; A61M 2205/3368; A61M 2205/3327

(57) ABSTRACT

The present invention patent relates to lighting lamps composed of LEDs which emit monochromatic or colored light and can produce, by interaction mechanisms and technology-supported narration models, behavioral changes in humans and other living beings by displaying texts, graphics or motion pictures using the light, and are previously programmed as encapsulated narrations which can be triggered by the users, providing novel aesthetic experiences. These aesthetic experiences are useful for managing anxiety, sleep cycles, changing habits, providing different forms of perceiving the environment, improving relationships, improving communication, improving self-esteem, the utilization of time, the regulation of the circadian cycle, decision-making on the basis of climate-relevant information, the analysis of activity in the environment, the awareness of consumption costs. This is possible because these lamps can operate at different light intensities, use biometrics, environment sensors and communication devices, and provide an interface for humans and other living beings with the technology.

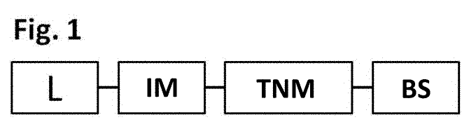

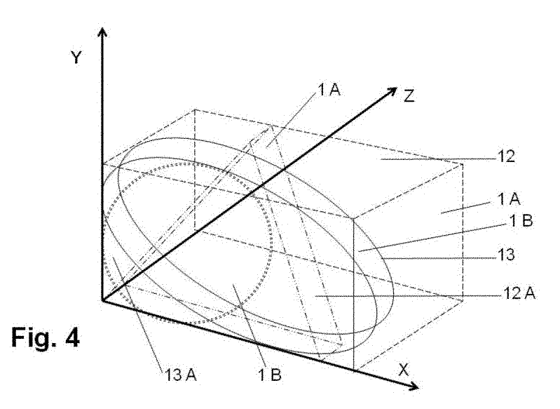

The present invention relates to a variety of monochromatic and colored lighting lamps (L) of FIGS. 1, 2, 3, 6, 9 and 10, comprised by an LED array (in different shape and arrangement). These lamps can be disposed in different proximal or distal contexts, of varying shapes, sized and geometries (12), (12A), (13), (13A) of FIG. 4 among others, capable of generating novel aesthetic experiences through interactive devices in external and internal interfaces (operative and control parts), so as to generate behavioral and experiential changes of the autotelic type, with benefits associated to the satisfaction of people and human beings (6) of FIG. 3 and other living beings such as animals and plants (7) of FIG. 3. These aesthetic experiences are the materialization of psychological wellness needs and desires to achieve them.

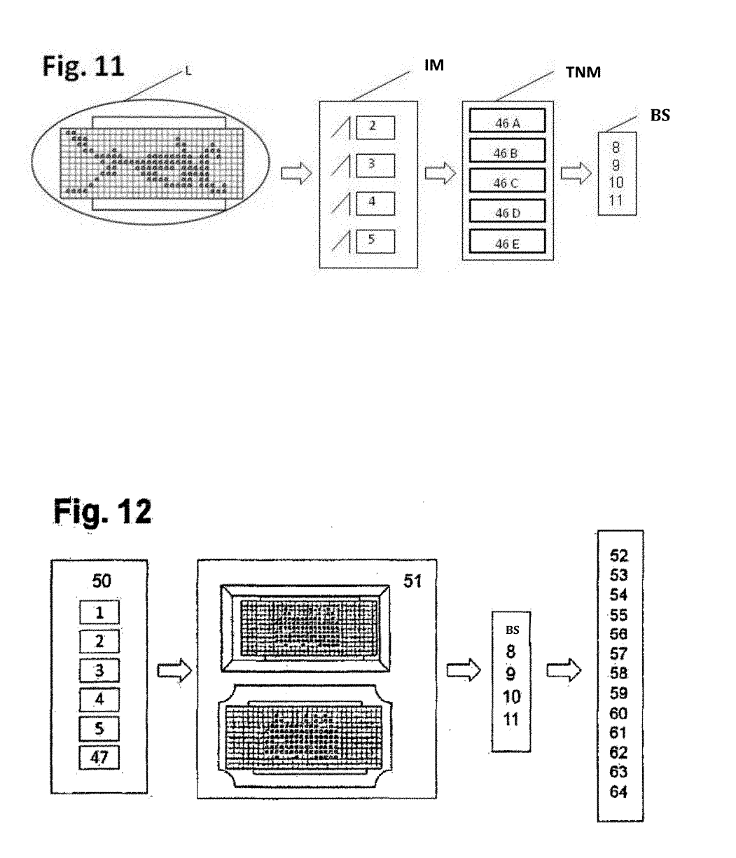

Such novel behaviors and experiences are carried out due to the combination of the interaction mechanisms (IM) of FIGS. 1 and 11, which allow the physical and electronic functioning of the lamps, with the technological narrative models (TNM) of FIGS. 1 and 11, which generate the activation of several situations, with the behavioral systems (BS) of FIGS. 1 and 1 as facilitators of the new experiences at mathematical level, to achieve: improvements in the affective relations among people, improvement in self-esteem, an adequate positive orientation of the objects in spaces according to its north orientation, environmental changes and experiences for leveling objects at home, orientation aids in individuals, adequate planning of daily activities, money saving and consuming costs awareness, treatment of fear and other childhood disorders, regulation of the cardiac cycle, anxiety management, improvement in health conditions, communication, safety, entertainment, reflection, spatiality and much more, which are achieved due to the lamps treating the aspects of affectivity through luminance, since these allow the expression of metaphysic and poetic relations with artefacts, through the deployment of texts and images by modulating the light intensity.

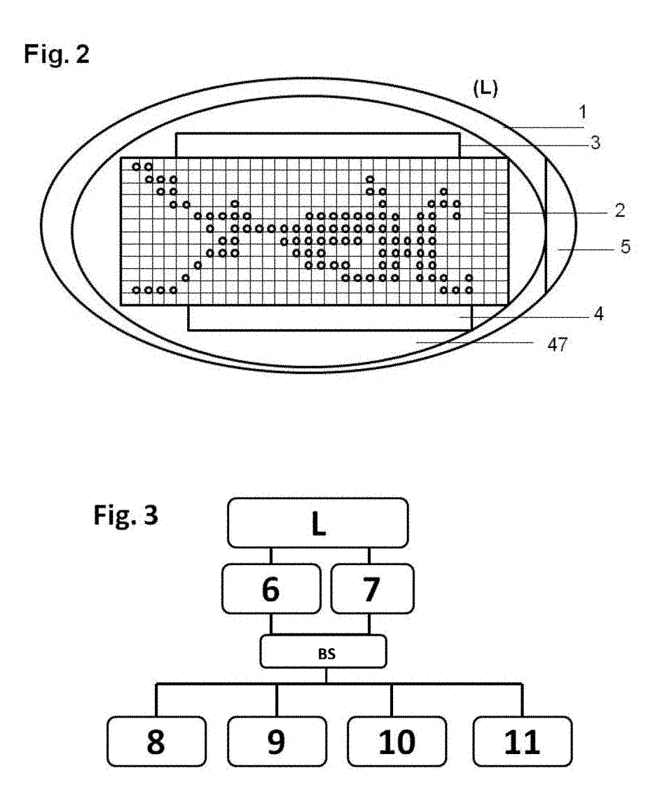

The lamps proposed in the present document, are comprised firstly by four interaction mechanisms (IM) of FIGS. 1 and 11, called LED screens (2) of FIGS. 2, 5, 6, 11 and 12, actuator (3) of FIGS. 2, 6, 7, 10, 11 and 12, controller (4) of FIGS. 2, 6, 7, 9, 10, 11 and 12 and the sensor (5) of FIGS. 2, 6, 7, 8, 11 and 12; all of which are disposed within the outer structure protection subsystem (1) of FIGS. 2, 4 and 12 and (1A) and (1B) of FIG. 4.

Such four interaction mechanisms activate, secondly, the five technological narrative models (TNM) of FIGS. 1 and 11 called fictitious (46A) of FIG. 11, interpretative (46B) of FIG. 11, interpretative with other people (46C) of FIG. 11, programmable (46D) of FIG. 11 and combinatorial (46E) of FIG. 11. And as a result, from the activation of the TNM, the behavioral systems (BS) of FIGS. 11 and 12 are developed, which are called biometrical (8) of FIGS. 3, 11 and 12, environmental (9) of FIGS. 3, 11 and 12, communicational (10) of FIGS. 3, 11 and 12, and experiential (11) of FIGS. 3, 11 and 12.

The interaction mechanisms (IM) of FIGS. 1, 5 and 11, are determined to work the physical and electronic functioning of the lamps and are the activators of the aesthetic experiences (technological narrative models—TNM), each fulfilling with a determined function defined as follow: in the case of the LED screen (2) of FIGS. 2, 5 and 6, the light emitting diodes are arranged in networks and in a determined number, according to the narrative. These can be coated of VMQ y PVMQ type silicon translucid elastomers (carbon free, good resistance to temperature, good electrical insulation, self-extinguishing). These light diodes are arranged in a printed circuit containing all the control electronics, which card constitutes in situ the screen as the lighting source. This arrangement may vary in size on the PLCC surface mounting with integrated reflector.

Each of the LEDs of the network, can be independently controlled in 4096 steps, with PMW (pulse width modulation) color or grayscale with 6-bit point correction, whereby each LED can be independently calibrated in case of existing manufacturing variations. The above allows texts and graphs in grayscale to be deployed if desired, through the control of the lighting intensity.

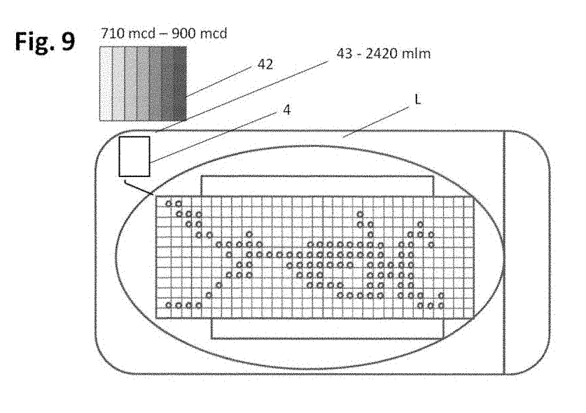

The visibility angle of each LED at 50% is 120 degrees and these emit monochromatic or color light, as currently may occur for the lighting of control boards, outdoor screens and reading lamps among others. It is also required that each LED has an operation temperature and storage ranging from −40° C. to 100° C., with forward current of 20 mA and an average forward voltage of 3.2 V. Its luminous intensity is 710 mcd and 900 mcd (42) of FIG. 9, with typical luminous flux of 2420 mlm (43) of FIG. 9.

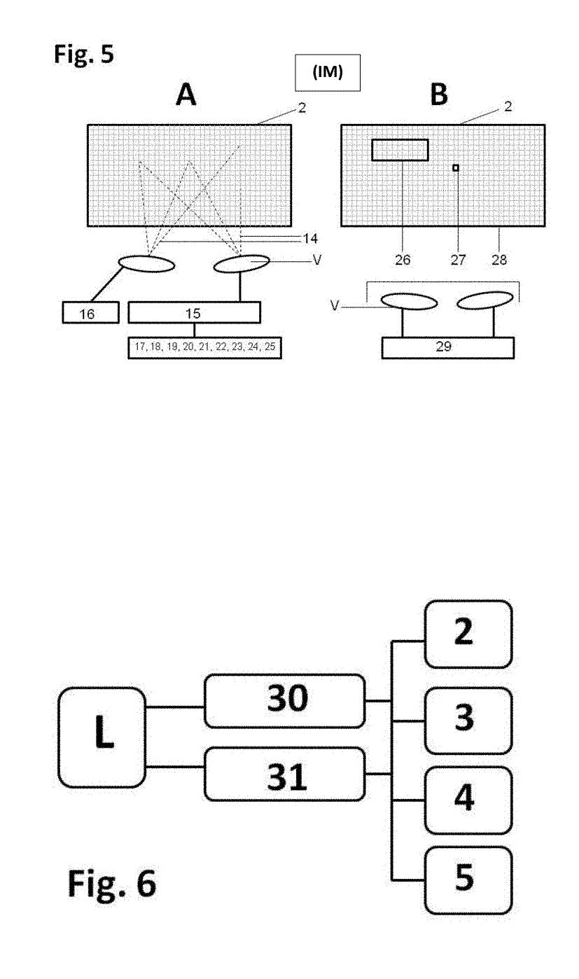

Such a screen (9) of FIGS. 2, 5 and 6, which is the fundamental element or factor for communication (10) of FIG. 3, allows texts or composition of well-founded graphics among others to be deployed by geometric or chromatic codes, besides of moving images which can be retinal or of previously programmed videos, if these are of the retinal type (A) of FIG. 5, corresponds to an apparent movement perception and if these are of previously programmed videos (B) of FIG. 5, corresponds to an animation of images playback.

In both cases (A and B), the image design in the LED screen takes into consideration the direct visual perception (14) of FIG. 5 in relation to way the brain perceives the information from the referenced and previously programmed imagen and the way it is processed and encoded through two reference systems, about the information entering through the eyes (V) of FIG. 5, by its visual addressing (14) of FIG. 5, called oculocentric reference (referenced by each eye) (16) of FIG. 5 and egocentric reference (common axis of both eyes) (15) of FIG. 5, for which the visual principles are involved.

The retinal movement images (A) of FIG. 5, are determined by the scale and ratio (17) of FIG. 5, the composition (18) of FIG. 5, the color scales (19) of FIG. 5, the contrast and size (20) of FIG. 5, the elements arrangement (21) of FIG. 5, the rhythm (22) of FIG. 5, the complementary colors management (23) of FIG. 5, the size and quality (24) of FIG. 5 and the movement by repetition (25) of FIG. 25 of FIG. 5 and for the previously programmed videos (B) of FIG. 5, the visual principals involved are those related to resolution (26) of FIG. 5 which depends on the amount of height and width pixels of the screenshot also called bit stream, the algorithmic compression (27) of FIG. 5, which depends of the application and the format (28) of FIG. 5, which can be determined through MPGE, MOV (associated to dynamism), AVI (audio and video interleave) or DIVX (combination of formats) files, and finally the video proportionality (29) of FIG. 5 is managed.

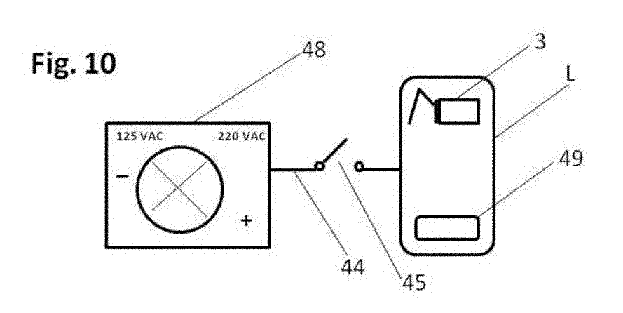

For the second case of the interaction mechanisms, we have the actuator (3) of FIGS. 2, 6 and 10 that is a type of button which allows to control the power feed to the system for turning on/off as opening and closure (45) of FIG. 10, this connection can be carried out among others, through power line-type cables (44) of FIG. 10, so as to bring the power feed (48) of FIG. 10 (which can be commuted with high energy efficiency) from 125 VAC to 220 VAC, also being able to be converted to 5 V through an internal transformer (49) of FIG. 10, with possibilities of connection of the lamps to USB ports.

The third interaction mechanism is associated to the controller (4) of FIGS. 2, 6, 7, 9 and 11, “which can be formed by sum union where the input and output signals are compared” Michael P. Groover, which allow to handle, in the case of lamps, the light intensity (42) of FIG. 42, the programming conditions and to work on the technical factors of the narratives. For the present case, four types of actions in the controllers can be used, which are called control (calling all or nothing), proportional control (acts by amplifying), derivative (anticipates errors and provides quick responses to changes) and integral (for the combination of control actions).

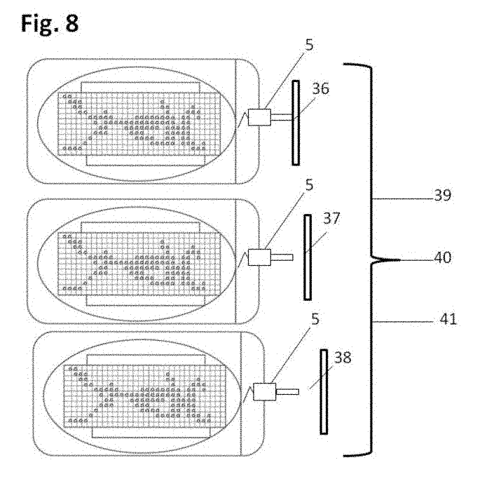

The fourth interaction mechanism is called sensor (5) of FIGS. 2, 6, 7, 8 and 11 which is arranged for the mathematical or statistic record of recognition of behavioral situations (BS) of FIGS. 1, 3 and 11, which can be tactile (36) of FIG. 8 which respond to contact forces, diverse (37) of FIG. 8 which measure factors such as temperature and pressure among others, and proximity (38) of FIG. 8, to indicate proximity measures.

The recognition by the sensor aided by the rest of the interaction mechanisms (IM) of FIGS. 1, 6 and 11, allow the “detonators” (technological narrative models—TNM) to act so as to determine the behavioral situations (BS) which for the present case are the biometric (8) of FIGS. 3 and 11, which allow the recognition and measurement of physical features of the individual (39) of FIG. 8, behavioral (40) of FIG. 8 and physiological (41) of FIG. 8 such as breathing, heart rate, body temperature and sweating among other; the environmental (9) of FIGS. 3 and 11 which allow the recognition and measurement of chemical, physical and biological conditions of the environment; the communicational (10) of FIGS. 3 and 11 for deployment of texts or graphic composition based on geometric and chromatic codes, which area achieved due to the LED screen (2) of FIGS. 2, 5 and 6; and the experiential (11) of FIGS. 3 and 11 for measuring the light intensity (42) of FIG. 9 and the light flux (43) of FIG. 9 achieved due to the controllers (4) of FIGS. 2, 6, 7, 9 and 11.

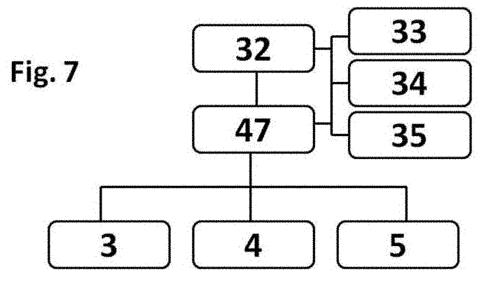

Both the LED screen (2) and the actuator (3), as well as the controller (4) and the sensor (5) entirely (which are jointly in the command and control operational part (30) and (31) respectively pertaining to FIG. 6) form the control circuit (47) of FIGS. 2 and 7, for regulating the information on the screen (2) whereby the limit (33), the system speed (34) of FIG. 7 and the stability (35) of FIG. 7 are determined in the lamp system. It should be understood that the control circuit (47) can properly work due to a “relation system” (32) called limited frequency which articulates (33), (34) and (35) with (3), (4) and (5) of FIG. 7.

All the components of the lamps proposed herein, are also enclosed in a protection subsystem or “case” which may vary according to the behavioral systems (BS) and to the context in which the activity is developed, which subsystem also encloses the interaction mechanisms (IM) of FIG. 2 (LED screen (2), actuator (3), controller (4), sensor (5) and control circuit (47)); this subsystem is called “outer structure protection subsystem” (1) of FIG. 2, which configuration can be constructed in polymeric materials both thermostable and thermoplastic, or if required, in thermostable or thermoplastic elastomers, or in Biopolymers in the desired classification, all under the ASTM D 883 standard of 2007, with the obligation to include in its composition, hydrophobic and dielectric additives with properties of connective heat adaptation and resistance of at least 1 cal/cm2, in order to protect the user from electric shock in variable contexts according to humidity levels (the latter not being applicable to Biopolymers). However, when constructing the lamps, other materials can be proposed, such as pulps with protective additives or in any ferrous metal.

Geometrically, the shape of the lamps must be adaptable according to the variations of the proximal or distal context, whereby these can be polygonal, included in the groups of polyhedrons (1A) or (12A) and (12) of FIG. 4 and non-polyhedrons or of round geometric bodies (1B) or (13A) and (13), whether in the orthohedrons sub-classification, such as prisms or pyramids for the first one, or spherical, cylindrical or cones for the second one.

These polygonal geometries of the outer structure protection subsystems (1) of the lamps, have two types of adaptations; on one hand the aesthetic which depends on the analysis made by the designer of the user coding in relation to the narrative, and on the other hand, according to the area generated by the number of LEDs arranged in the assembly also in relation to the narrative. However, there are two common elements to all the formal and geometric variations of the LED lamps and correspond to the ignition and unleashing controls of the narrative “enclosed” in the lamp.

These polyhedral (1A) or non-polyhedral (1B) or of round geometric body structures allow the interaction mechanisms (IM) and all the controls to be clearly arranged and organized, for the functioning of the technological narrative models (TNM), which, as mentioned above, are the detonators (activators) of the narratives, FIGS. 1 and 11.

These technological narrative models (TNM) are called fictitious (46A) of FIG. 11, interpretative (46B) of FIG. 11, interpretative with other people (46C) of FIG. 11, programmable (46D) of FIG. 11 and combinatorial (46E), which function as follow: the fictitious (46A) regulate the light intensity through the controller (4) and the actuator (3) allowing thereby the detonation (activation) of any narrative; the interpretative (46B) which makes the narrative to be associated to the communication, also working the light intensity, and the process unleashing, which is achieved according to the LED screen (2), the controller (4) and the actuator (3).

The interpretative with other people (46C) control the light intensity, so as to allow the relation which other people (6) of FIG. 3 and with other beings (7) of FIG. 3, through the interpretation of mathematical values of these beings. These also allow to feel the environment in the different narratives, all of which is achieved associated with the sensor (5), the actuator (3) and the controller (4).

The programmable (46D) allow the management of times, type of movements and also the light intensity, along with the sensors (5), the controller (4) and the actuator (3).

For the case of the combinatorial (46E) of FIG. 11, it combines the features of the previous four (46A), (46B), (46C) and (46D), along with the four interaction mechanisms (IM); LED screen (2), actuator (3), controller (4) and sensor (5).

Finally, although the interaction mechanisms—IM (LED screen (2), actuator (3), controller (4) and sensor (5)) with the control circuit (47) and the outer structure protection subsystem (1), are present as long as they have the same electronic and material functions (50) of FIG. 12, the formal and structural variations generated from the lamps (51) of FIG. 12 will depend on the behavioral systems (BS), so as to achieved the new experiences which in its structural relation can be called among many others: Circadian light (52), Counting sheep light (53), emoji light (54), dream light (55), weather light (56), daily planner light (57), heartbeat light, (58), pet light (59), synth light (60), pong light (61), moon light (62), weight light (63), light O'matic (64) all of FIG. 12 and many more possibilities, which names by themselves talk about the type of narrative they are arranged to.

1. Lighting lamps generating novel aesthetic experiences in humans and other beings, through the use of physical interactive systems characterized by comprising as interaction mechanisms (IM):
an outer structure protection subsystem which groups all devices of a system, the system comprising:
a reading device for reading physical, chemical and biological agents;
a controlling device for controlling driving of the reading device;
an activating device for activating and deactivating power;
a voltage regulating device of switched current as feeder for the activating device; and
a printed circuit which groups a network of light emitting diodes (LEDs) or monochromatic or color LED screen activated by the activating device and the controlling device.

2. Lighting lamps generating novel aesthetic experiences in humans and other beings, through the use of physical interactive systems, characterized according to claim 1, in that the outer structure protection subsystem is a flexible exoskeleton in which the reading device, the controlling device, the activating device, the voltage regulating device, and the printed circuit or LED screen are coupled to.

3. Lighting lamps generating novel aesthetic experiences in humans and other beings, through the use of physical interactive systems, characterized according to claim 1, in that the reading device is a multiple reading sensor.

4. Lighting lamps generating novel aesthetic experiences in humans and other beings, through the use of physical interactive systems, characterized according to claim 1, in that the reading device is a temperature, pressure, distance and objects sensor.

5. Lighting lamps generating novel aesthetic experiences in humans and other beings, through the use of physical interactive systems, characterized according to claim 1, in that the controlling device is an electric transformation controller.

6. Lighting lamps generating novel aesthetic experiences in humans and other beings, through the use of physical interactive systems, characterized according to claim 1, in that the controlling device transforms values read by the reading device, in electric pulses.

7. Lighting lamps generating novel aesthetic experiences in humans and other beings, through the use of physical interactive systems, characterized according to claim 1, in that the network groups diodes, and is a PLCC surface LED screen with an integrated reflector.

8. Lighting lamps generating novel aesthetic experiences in humans and other beings, through the use of physical interactive systems, characterized according to claim 1, in that the LED screen converts values of physical, chemical and biological agents translated by the controlling device in images.

9. Lighting lamps generating novel aesthetic experiences in humans and other beings, through the use of physical interactive systems, characterized according to claim 1, in that the voltage regulating device is a button actuator which activates and deactivates power from the interaction mechanisms (IM), according to values of the printed circuit of the network or the LED screen.

10. Lighting lamps generating novel aesthetic experiences in humans and other beings, through the use of physical interactive systems, characterized according to claims 1 and 9, in that the voltage regulating device processes voltages from 125 VAC to 220 VAC and conversion to 5V.

11. Lighting lamps generating novel aesthetic experiences in humans and other beings, through the use of physical interactive systems, characterized according to claim 3, in that the LED screen, an integrated reflector, the controlling device, and the reading device, altogether determine aesthetic thresholds of the lamps.
